(19) United States
(12) Patent Application Publication
(10) Pub. No.: US 2009/0270789 A1
(43) Pub. Date: Oct. 29, 2009

(54) SUCTION DOME FOR ATRAUMATICALLY GRASPING OR MANIPULATING TISSUE

(76) Inventors: George W. Maxymiv, Roanoke, VA (US); Mark Gustafson, Salem, VA (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 12/296,944

(22) PCT Filed: Apr. 13, 2007
(86) PCT No.: PCT/US07/09044

§ 371 Date: Oct. 13, 2008

Related U.S. Application Data
(60) Provisional application No. 60/791,897, filed on Apr. 14, 2006.

(51) Int. Cl. A61B 17/285; A61B 17/28; A61M 5/00; A61M 1/00

(52) U.S. Cl. 604/22; 606/207; 604/272

(57) ABSTRACT

The present invention provides a suction dome for atraumatically grasping, manipulating, and/or extracting tissues. The suction dome may be used with a surgical instrument having a suction channel. Such instruments include, for example: forceps, laparoscopes, endoscopes, bronchoscopes, and catheters. The suction dome comprises an outer wall non-permeable membrane and a tissue-engaging permeable membrane base. The non-permeable membrane may be supported by a plurality of expanding arms and defines a chamber in communication with the suction channel of the instrument.

### CROSS-REFERENCE TO RELATED APPLICATIONS

This application relies on and claims the benefit of the filing date of U.S. provisional patent application No. 60/791,897, filed 14 Apr. 2006, the entire disclosure of which is hereby incorporated herein by reference in its entirety.

### SUMMARY OF THE INVENTION

1. Field of the Invention

The present invention is generally directed to a surgical device, and more particularly to a suction dome for atraumatically grasping and/or manipulating tissue.

2. Background of the Invention

Surgical forceps are used for grasping, retracting, and/or dissecting tissues during surgical procedures. In essence, forceps act as an extension of a surgeon's hands in limited areas of access. Forceps may be used for a variety of purposes, from grasping tumors for dissection to moving and manipulating intervening tissues.

Although there are many configurations, conventional surgical forceps are generally characterized by two opposing fingers, or jaws, which are moved together in order to grasp tissue between them. These types of forceps operate by applying inward compression forces on the tissue until it may be lifted or manipulated without slipping. In practice, however, such mechanical forceps exhibit poor tissue grasping and holding capabilities. One reason for this is because they require a certain amount of friction to exist between the jaws of the forceps and the tissue surface. The less friction that is present, the more inward forces must be exerted before a tissue can be successfully lifted or manipulated. On the other hand, the more a tissue is compressed, the more likely it is to be injured or to rupture. This is a particular concern with fluid-filled cysts, especially where uncertainty exists with respect to the wall thickness and resistance to rupture.

In order to obtain a better grasp on tissue with less compression force, some forceps include either sharp teeth or serrations in the jaws. Examples of such teeth include the Richard Wolf 8385.10, 8385.13, and 8383.471 type grasping forceps. Although teeth and serrations may help to prevent slipping, they can also cause trauma by way of puncturing or lacerating tissue. Such punctures in tissues or tumors may increase the risk of patient infection or allow undesirable spreading of malignant tumor cells. At the very least, such teeth cause unnecessary damage to target tissues, and especially tissues that must be grasped repeatedly or require a great deal of manipulation.

Another problem associated with conventional surgical forceps is that of a target tissue “bouncing” away from the tip of the forceps as the surgeon attempts to grasp the tissue. This occurs frequently with large, smooth, and/or resilient, or hard firm tissues (such as glands, organs, cysts and parenchymal tissues). One factor that can compound this problem is the limited opening width of thejaws. If a target tissue is larger than the opening in the forceps, and/or if sufficient friction between the forceps and tissue is not present, “away”-ward forces might override compression forces “normal” to the surface and the tissue will bounce away from the forceps. In addition, certain tissue types can also present a challenge to grasp without causing injury. For example, ovarian tissue can be difficult to grasp and control without tearing and bleeding. Currently, grasping ovarian cysts with conventional forceps without rupture is very difficult. Any occurrence of rupture defeats the purpose of a cystectomy (making it more difficult to remove). In addition, other tissues, such as the spleen, pose serious risks to the patient if ruptured. Thus, not only is such “bouncing” an inconvenience to the surgeon, but may consume valuable time during a procedure and increase overall health risks to the patient.

Other instruments for engaging and holding tissues have been devised that utilize suction to impose traction on tissues instead of compression. However some devices, for example as disclosed in U.S. Pat. No. 3,896,810, use suction structures composed of rigid materials, such as metal. These structures are not very flexible or conformable to different tissue surfaces, and thus are not suited for engaging irregularly shaped, and/or delicate, tissues. Other devices, for example as disclosed in U.S. Pat. No. 6,641,575, use somewhat flexible suction cups. However, these still only physically engage the tissue around the periphery of the cup. Thus, problems occur if the vacuum seal around the periphery is broken (e.g., due to poor contact with an irregular tissue surface). In this case, the suction force on tissue is significantly weakened and compromised. Such weakened contact could allow undesirable fluids to escape (or enter) the area. This result could increase risk of infection or spreading of malignant tumor cells.

What is needed, therefore, is a device that is able to grasp tissue in a reliable manner while maintaining tissue integrity. In addition, what is needed is a device that provides improved tissue grasping and manipulating capabilities in a less traumatic manner. The suction dome of the present invention is able to meet these needs and, at the same time, provides a significant improvement over the prior art. These and other advantages of the present invention will become apparent from the disclosure herein.

### SUMMARY OF THE INVENTION

In a first aspect, the present invention provides an expandable suction dome for use at the distal end of a surgical instrument for atraumatically grasping and/or manipulating target tissue. The invention also provides a surgical instrument including a suction dome comprising: a non-permeable outer membrane defining an inner chamber; and a permeable tissue-engaging membrane extending across the base of the chamber. In general, the present invention provides a surgical instrument, such as a forceps or laparoscope, having a longitudinal suction channel and an optional needle channel. The suction dome is translated in a collapsed state through the suction channel and is operatively deployed after exiting the distal end of the instrument.

The outer membrane of the suction dome may be coupled to a plurality of expandable arms and is sufficiently supported to withstand internal vacuum pressures and external bodily pressures or artificially produced pressures when deployed. A primary purpose of the non-perneable outer membrane is to help maintain a vacuum-tight seal between the dome and tissue. The tissue-engaging membrane, on the other hand, is sufficiently permeable so as to allow the vacuum to be evenly distributed over the entire surface in contact with the tissue. In this way, the suction dome is able to atraumatically grasp and manipulate tissue by cupping it with the permeable membrane. The supporting arms in the dome wall also act as graspers and hold the tissue as the wall of the dome is retracted back into the sheath. An optional needle may also be inserted through a needle port of the instrument for puncturing, irrigating, and/or aspirating tissues with fluids or medications. The target tissue may include any tissue, including but not limited to: tumors, cysts, organs, and glands. In addition, although a forceps and laparoscope have been mentioned by way of example, it is to be understood that any instrument including, but not limited to: endoscopes, bronchoscopes, and catheters may also be used with the suction dome.

### BRIEF DESCRIPTION OF THE DRAWINGS

FIG. 1 is a perspective view of a suction dome showing internal components and constructed in accordance with the teachings of the present invention.

FIG. 2A is an isometric view of a surgical instrument comprising a suction dome, sheath, and handle according to one embodiment.

FIG. 2B is a perspective cut-away view of the suction dome with respect to the distal end of the surgical instrument shown in FIG. 2A.

FIG. 2C is an exemplary cross-sectional illustration of the surgical instrument taken along the middle portion of the instrument.

FIG. 3 is an exemplary isometric view of the suction and aspiration ports at the proximal end of the surgical instrument.

FIG. 4Aa is an isometric view of the suction dome in a partially collapsed state according to another embodiment of the present invention.

FIG. 4B is an isometric view of the suction dome in a collapsed state prior to deployment and/or retraction.

FIG. 5A illustrates a general laparoscopic procedure in which the suction dome may be used.

FIG. 5B illustrates aspiration of a cyst using a needle introduced through the suction dome.

FIGS. 6A and 6B illustrate removal of a cyst through an abdominal cavity using the suction dome.

FIG. 7 is another isometric view of the suction dome showing the permeable membrane, and perforations therein, constructed in accordance with the teachings of the present invention.

### DETAILED DESCRIPTION OF EMBODIMENTS OF THE INVENTION

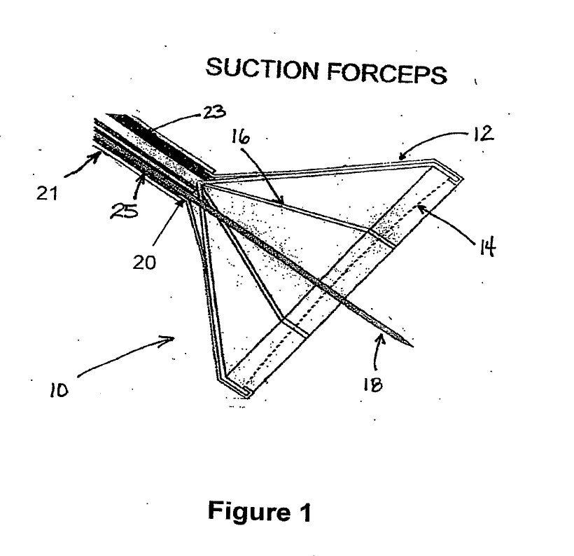

FIG. 1 shows a representative view of a suction dome according to the present invention. This figure shows the suction dome (10) at the distal end of an instrument having an outer sheath (21) and an inner sheath (20) defining a suction channel (23) according to one embodiment of the present invention. In this figure, the suction dome (10) is shown in a deployed state and has an inverted umbrella-like shape defining an internal chamber. The suction dome (10) is comprised of: a non-permeable, outer membrane (12) forming the outer wall; and a semi-permeable tissue-engaging membrane (14) extending across the base of the chamber for securely and uniformly engaging a surface area of tissue. In this embodiment, the outer membrane (12) is integral with, or connected to, the distal portion of the inner sheath (20). Preferably, the inner sheath (20) is slidable within the outer sheath (21) for retraction and extension of the dome. The suction dome (10) may also include several arms (16) coupled to the outer membrane (12) for support and deployment thereof. Arms (16) may be composed of a resilient and/or memory material such that they expand automatically as the dome (10) is extended out of the outer sheath (21).

The outer wall (12) and inner sheath (20) of the dome are constructed of a non-permeable membrane, which can be made out of any suitable material. It is desirable to have a non-permeable membrane to maintain sufficient vacuum between the suction channel (23) and the base membrane (14). The outer membrane (12) is also sufficiently supported (e.g., by arms (16)) so as not to collapse under negative vacuum pressure within the dome or external bodily pressures. The outer membrane (12) is sufficiently pliable so as to be collapsed when not in use. Suitable materials for the outer membrane (12) and/or inner sheath (20) include, but are not necessarily limited to: plastic, polyethylene, silicone, rubber, or combinations thereof, etc. In addition, it may be desirable for the outer membrane (12) (or a portion thereof) to be transparent so as to allow visualization of the tissue underneath or of the procedure as it is carried out. It is also desirable for a vacuum-tight seal to be made between the suction dome (10) and the tissue to prevent any gas or fluid from entering and/or escaping from the interior of the dome. Target tissues may include any type of bodily tissue, including, but not limited to organs, glands, cysts, and tumors. Examples may include ovarian cysts, gall bladder, ectopic pregnancy, etc.

The base of the dome, or tissue-engaging membrane (14) is constructed of a semi-permeable membrane. This type of membrane can be used to allow negative air pressure (as applied through the suction channel) to be evenly distributed over its surface area and transmitted onto the surface of the tissue in contact. When suction is applied, membrane (14) allows negative pressure to pass directly through perforations, or holes, therein. In this way, negative pressure is substantially uniformly applied to the tissue from the base surface of the dome (10) over the entire tissue surface area in contact. The diameter of membrane (14) should be large enough to apply to an adequate surface area of the tissue. The membrane (14) is preferably composed of a flexible and pliable material that enables the target tissue to be closely cupped therein as suction is applied. Thus, the dome is able to more securely engage the target tissue and hold it in place with minimal, or no, trauma to the tissue itself. Moreover, close contact of membrane (14) with delicate tissue surfaces such as fluid-filled cysts may serve as additional reinforcement (as they are penetrated by needles), thereby reducing rupture or tearing associated with thin tissues. The permeable membrane (14) is sufficiently pliable so as to be collapsed when not in use. Suitable materials for the tissue-engaging membrane (14) include, but are not limited to: plastic, polyethylene, silicone, rubber, or combinations thereof, etc. It is possible that part or all of the permeable and non-permeable membranes may be composed (in whole, or in part) of the same, or different, materials. If the same materials are used, the tissue-engaging membrane (14) may still be somewhat more flexible than the outer membrane (12) (e.g., by virtue of the perforations therein). It is also to be understood that membranes (12) and (14) may comprise one or more layers of material.

The permeability in membrane (14) may be achieved in any number of ways, the particular way not being critical to practice of the invention. For example, it can be by a plurality of discrete, spaced apart, holes therein or may be intrinsic to the material itself (such as with a fabric mesh). In addition, irregularly shaped target tissues, for example, may be more reliably engaged. The size, number and spacing of the perforations in the material may also vary depending upon the type of target tissue and the necessary amount of suction.

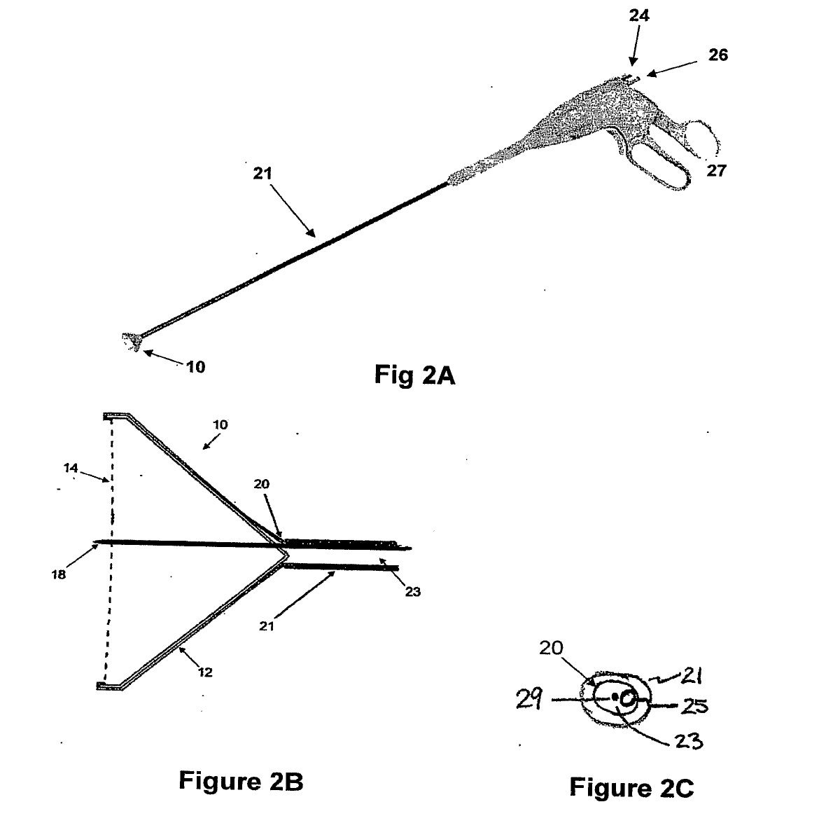

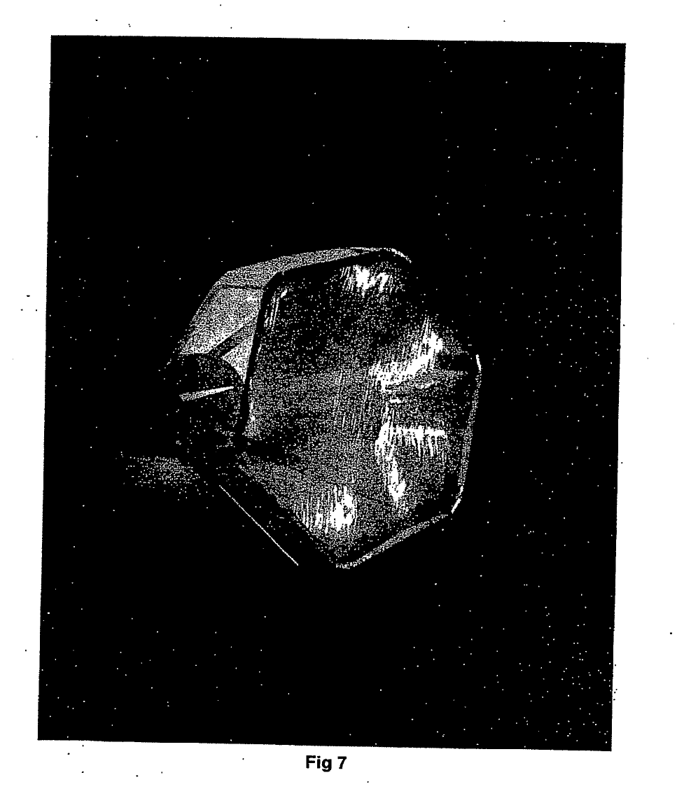

In operation, the outer membrane (12) and/or inner sheath (20) are typically operably coupled to arms (16) such that outward extension of the arms (16) at the distal end of the instrument causes the suction dome (10) to be deployed as shown in FIG. 1. The arms (16) may open and close the dome (10) as they are extended beyond, or retracted within, the outer sheath (21). Arms (16) may be comprised of a resilient and/or shape-memory material such that they automatically expand upon extension from outer sheath (21). In addition, the outer membrane (12) is integral with, or connected to, the distal end of inner sheath (20) and may be extended or retracted e.g., via a proximal retracting mechanism (27, shown in FIG. 2A) operably coupled to the inner sheath (20). In an alternative embodiment, the arms (16) may be controlled, e.g., by a longitudinal support wire, or actuation cable (not shown) and operably extended or collapsed by movement of a proximal retracting control mechanism (27, shown in FIG. 2). Additionally or alternatively, the arms (16) may be composed of a preformed material that automatically extends and collapses upon exit, or entry, of the outer sheath. As shown in FIG. 1 and FIG. 7, the arms (16) may also include inwardly-folding joints at the distal-most portions. Such distal joints on the arms (16) fold inwardly as the dome is retracted, thereby more firmly grasping and securing tissue therein. FIG. 7 also illustrates the plurality of spaced apart perforations, or holes, in the permeable membrane.

Another advantage of the suction dome according to embodiments of the present invention is increased maneuverability of tissue. For example, when a tissue (such as a cyst) is dissected and freed, the suction dome (10) may be used to control movement of the tissue in vertical, horizontal and rotational directions. For increased maneuverability, the suction dome may be used in conjunction with a forceps having an articulating handle and sheath.

Also shown in FIG. 1 is an optional needle or cannula (18) which may be introduced through a needle channel (25) in the instrument. At the distal end of the instrument, the needle (18) may continue to be advanced through the interior of suction dome (10) and through perforations in order to penetrate the target tissue. Needle (18) can be used for puncturing, irrigating, and/or aspirating tissue. For example, if the tissue is a fluid-filled cyst, the needle (18) can be used for draining the contents of the cyst. Additionally or alternatively, the needle (18) may be used for introducing various fluids or medications to tissues.

FIG. 2A illustrates proximal, middle, and distal portions of an instrument used with the suction dome according to one embodiment. In the figure, the middle portion of the instrument comprises a flexible, or rigid, outer sheath (21) through which arm control mechanism, suction (23) and needle (25) channels extend longitudinally. Such outer sheaths for laparoscopy and other surgical tools are known in the art, and any suitable size sheath may be used according to the present invention. Typically, suitable diameters for the outer sheath (21) range from 3-20 mm, and preferably 3-10 mm, although other diameters may be used. For example, diameters between 10-20 mm may be used for resection of larger tissues such as gall bladder, appendix, myomata, etc. The working length of the instrument may be, for example, 240, 310 or 430 mm, although other lengths may be used. Working lengths of 240 mm are suitable for introduction through an accessory port and lengths of 310 and 430 mm are suitable for introduction through an accessory port or laparoscope. At the distal end of the instrument, the suction dome (10) is shown deployed. When a procedure is complete, the dome is retracted back into the sheath, for example by releasing a retraction control mechanism (27, discussed below).

Also at the proximal end of the instrument of FIG. 2A is a handle with an extraction/retraction control mechanism (27) operatively coupled to the suction dome (10) via inner sheath (20, shown in FIG. 2B). In an alternative embodiment, the retraction control mechanism (27) may be operatively coupled to the suction dome (10) via a support wire and/or an actuation cable (not shown), or any other conventional means, such as those known in the art. The retracting control mechanism (27) shown in FIG. 2A is a conventional scissor-like thumb and forefinger control; however, it should be understood that the retracting control mechanism (27) and/or handle could alternatively comprise other forms, such as a spring thumb ring and a friction stop. Additionally, instead of manual manipulation, the retraction control mechanism (27) may also be automated.

FIG. 2B illustrates a partial distal view of the suction dome (10) with respect to the interior of the instrument. Outer sheath (21) is shown as well as inner sheath (20) defining suction channel (23). FIG. 2C illustrates an exemplary cross-sectional view of the elements. In this figure, suction channel (23) is defined by inner sheath (20) disposed within outer sheath (21). In addition, optional components may also include: a support wire, or actuation cable (29), and a needle channel (25). It is to be understood that the arrangement of the internal components is provided only by way of example, and other arrangements may be possible.

The distal, middle and proximal portions of the instrument may be fixed or, alternatively, may be modular so as to improve ease of interchangeability with different sized lumens and suction domes. Such interchangeability helps to reduce replacement costs as well as cleaning and sterilization times. Current modular laparoscopes and forceps (including separate handles, mechanical jaw inserts and sheath tubes) include, for example: ConMed's DetachaTip™ System, SpeedLock™ Laparoscopic Instrumentation, and Richard Wolf modular/reusable forceps by Medical Instruments Corporation. For example, the suction dome of the present invention may be used with a conventional modular sheath and handle providing a vacuum channel and optionally a needle channel.

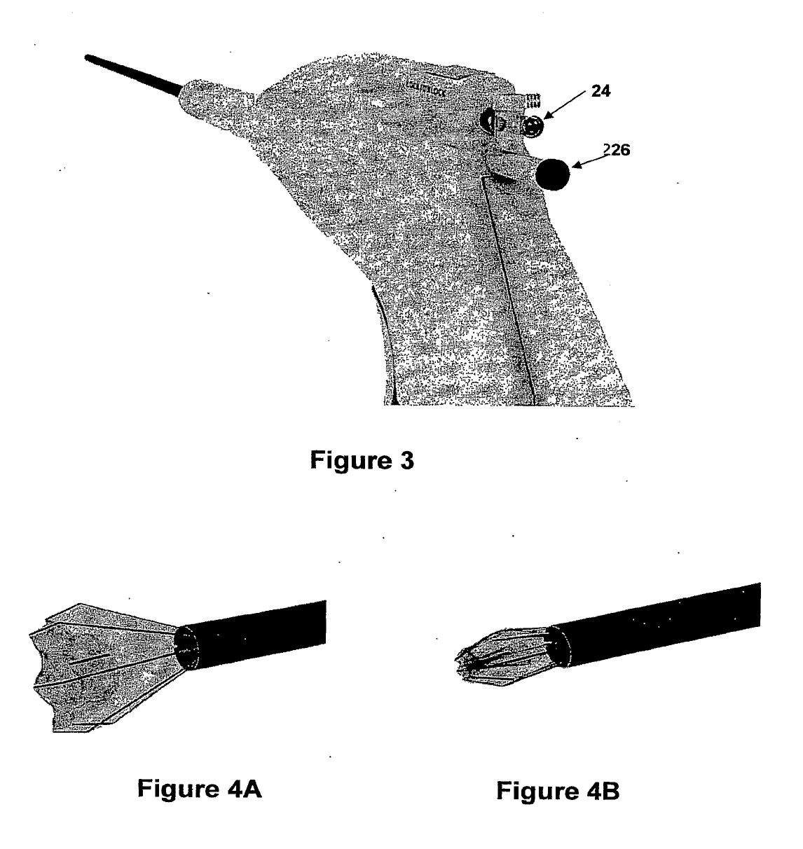

At the proximal end of the instrument shown in FIG. 3 are entry ports for vacuum (24) and needle access (26) for the suction channel (23, shown in FIGS. 2B and 2C) and needle channel (25, shown in FIGS. 2B and 2C). The suction entry port (24) may be coupled to any conventional source of suction (not shown) to provide a sufficient grasping force on the target tissue. Such sources include, but are not limited to: electro-mechanical pumps or manual pumps. The needle entry port (26) may be coupled to a separate conventional irrigation and/or aspiration source (not shown). Preferably, the needle access port (26) should be self-sealing to prevent loss of vacuum in the suction dome. The needle port may be sized to accept variable sized needles (not shown).

A needle, or cannula, (18, shown in FIG. 1) may be used with the device of the present invention. The needle or cannula is inserted through the needle port (26, shown in FIG. 3) and may be coupled to a conventional source of irrigation and/or aspiration e.g., to drain contents of the target area, or to introduce various fluids or medications. Although any needle (18) suitable for a particular surgical procedure may be used, preferably the needle (18) is a long cannula with a hard finished beveled tip. Use of a beveled or pointed tip helps to ease penetration of tissue and thin membranes and reduces risk of rupture. For example, the needle (18) may be a long cannula with a diameter of about 16-18 g. In another aspect, the needle (18) or cannula may also be composed of a disposable material (such as plastic) to avoid cross-contamination and to reduce sterilization times.

As illustrated in FIGS. 4A and 4B, the arms (16) may additionally include inwardly-folding joints at the distal portions. Such distal joints on the arms (16) fold inwardly as the dome (10) is retracted. FIG. 4A shows the suction dome (10) partially collapsed, and FIG. 4B shows the suction dome (10) collapsed prior to retraction and/or deployment. As the arms (16) come down from all sides, the tissue may thereby be grasped with greater power so as to pull it toward, or into, the instrument. For example, once firmly grasped by the suction dome (10) using the principles of the present invention, a cyst may be pulled into, or toward, the instrument along with the suction dome (10) and subsequently pulled out of the abdomen intact.

Although a dome-like shape has been ascribed to the outer membrane (12) when deployed, it is to be understood that, the shape and size of the suction dome (10) may vary according to the intended application. Suitable configurations may also include frustro-conical, elliptical as well as hemispherical shapes. While the size and diameter of the suction dome may vary according to the size and type of tissue to be grasped and/or manipulated, suitable diameters include 10-100 mm, and preferably around 20-30 mm.

### Example

### Laparoscopic Ovarian Cystectomy

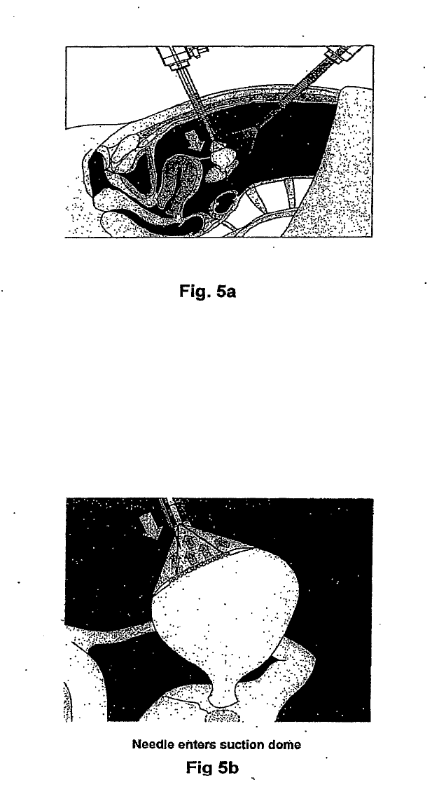

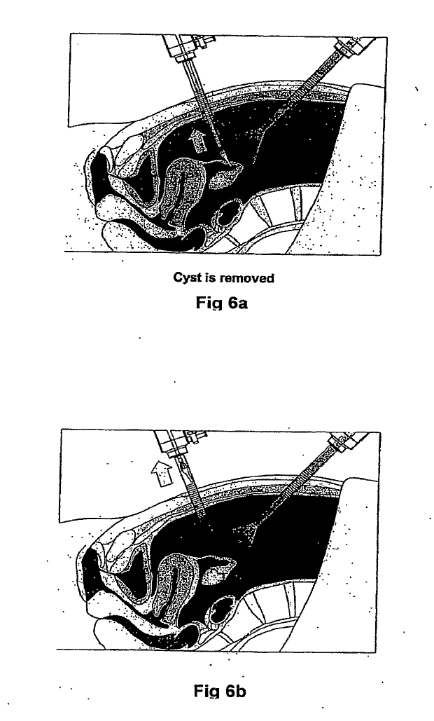

Laparoscopic ovarian cystectomy is a common surgical procedure. FIGS. 4A, 4B, 5A, and 5B illustrate such a laparoscopic procedure for draining and removing an ovarian cyst using the principles of the present invention. As with conventional laparoscopic surgery, lateral and/or umbilical incisions made using e.g., (5, 7 or 10 mm) dilating tip trocars to create entry sites. An endoscope and a 310 mm suction forceps are introduced into the abdomen through the entry sites. The abdomen is distended with carbon dioxide gas, where pressures in the abdomen should not exceed about 20 mm Hg. Under endoscopic observation, the suction forceps approaches the ovarian cyst. As the forceps draws near the cyst, the suction dome is deployed using retraction a control mechanism (not shown). A sufficient amount of suction is applied through the suction port to draw the cyst into reliable contact with the permeable membrane of the dome.

Once the cyst has been reliably grasped by the forceps, it is held in place while being dissected from the surrounding ovarian tissue using a secondary laparoscopic device. At this point, the cyst is able to be lifted and freely moved in any direction. To prepare the cyst for removal from the body, an aspiration needle is translated through a needle channel in the forceps, through the interior of the suction dome, and inserted into the cyst. The cyst is drained through the needle using conventional aspiration techniques.

Upon complete drainage, the outer membrane of the suction dome is collapsed (e.g., by closing support arms) around part of the cyst. Suction is maintained to retain good contact between the membrane and the tissue. The support arms in the outer membrane help grasp and secure the deflated cyst. The suction forceps is withdrawn through the access port and the cyst pulled out intact though the abdomen. Alternatively, the cyst may be placed into an endo-bag for deflation and subsequent removal.

While various preferred embodiments have been shown and described, it will be understood that there is no intent to limit the invention by such disclosure. Rather, the disclosure is intended to cover all modifications and alternative constructions falling within the spirit and scope of the invention as defined in the appended claims.

1. A suction dome for atraumatically engaging tissue at the distal end of a surgical instrument having a suction channel, the suction dome comprising:
a non-permeable, outer membrane defining a chamber therein, the chamber in communication with the suction channel of the instrument;
a flexible permeable membrane extending across the base of the chamber and configured to substantially uniformly engage a portion of target tissue when suction is applied through the chamber; and
the outer membrane supported by a plurality of expandable arms wherein the arms have inwardly-foldable distal joints capable of controllably grasping the target tissue within the chamber.

2. (canceled)

3. The suction dome of claim 1, wherein the non-permeable membrane, permeable membrane, and arms are collapsible to enable longitudinal translation of the suction dome through the suction channel of the instrument before, and/or after deployment.

4. (canceled)

5. The suction dome of claim 3, wherein the arms are operably coupled to a retracting control mechanism at the proximal end of the instrument.

6. The suction dome of claim 5, wherein the arms may be controllably expanded and collapsed by operation of the retracting control mechanism.

7. The suction dome of claim 1, wherein the permeable membrane comprises a plurality of spaced apart holes therein.

8. A surgical instrument comprising the suction dome of claim 1, the surgical instrument selected from one of: forceps, laparoscopes, endoscopes, bronchoscopes, and catheters.

9. The suction dome of claim 1, further comprising a needle operably insertable through the chamber and permeable membrane to puncture, irrigate and/or aspirate the target tissue.

10. A surgical instrument having a proximal and distal end, including:
a handle disposed at the proximal end, the handle having a suction port for communication with an external source of suction, and a port to allow insertion of an aspiration needle to reach a cyst for aspiration;
a longitudinal sheath coupled to the handle and having a suction channel therein, the suction channel being in communication with the suction port; and
a suction dome capable of being collapsed for longitudinal translation through the suction channel and deployed after exiting the distal end of the instrument, the suction dome comprising:
a non-permeable, outer membrane defining a chamber therein, the chamber in communication with the suction channel of the instrument; and
a flexible permeable membrane extending across the base of the chamber and configured to substantially uniformly engage a portion of target tissue when suction is applied through the chamber; and
the outer membrane supported by a plurality of expandable arms wherein the arms have inwardly-foldable distal joints capable of controllably grasping the target tissue within the chamber.

11. (canceled)

12. The surgical instrument of claim 10, wherein the non-permeable membrane, permeable membrane, and arms are collapsible to enable longitudinal retraction of the suction dome into the handle of the instrument before, and/or after deployment.

13. The surgical instrument of claim 12, wherein the arms include foldable distal joints which assist in grasping the tissue.

14. The surgical instrument of claim 12, wherein the arms are operably coupled to a retracting control mechanism at the proximal end of the instrument.

15. The surgical instrument of claim 14, wherein the arms may be controllably expanded and collapsed by operation of the retracting control mechanism.

16. The surgical instrument of claim 10, wherein the permeable membrane comprises a plurality of spaced apart holes therein.

17. The surgical instrument of claim 10, wherein the instrument is a:
forceps, laparoscope, endoscope, bronchoscope, or catheter.

18. The surgical instrument of claim 10, wherein the handle further includes a needle aspiration port, and the longitudinal sheath further includes a needle channel.

19. The surgical instrument of claim 18, further including an aspiration needle inserted through the needle channel and the suction dome to puncture, irrigate and/or aspirate the target tissue.

20. The surgical instrument of claim 10, wherein the handle, sheath and suction dome are of modular construction.

21. A method for atraumatically and reliably grasping and/or manipulating tissue with a suction dome in operative communication with a suction channel of a surgical instrument, the method comprising the steps of:
advancing a suction dome toward target tissue,
the suction dome comprising:
a non-permeable, outer membrane defining an inner chamber; and
a flexible, permeable membrane extending across the bottom of the chamber; and
a plurality of expandable arms supporting the outer membrane wherein the arms have inwardly-foldable distal joints capable of controllably gasping the target tissue within the chamber;
applying suction through the dome via the suction channel; and
substantially uniformly engaging a portion of the target tissue with the permeable membrane when suction is applied through the dome so as to atraumatically and reliably grasp and/or manipulate the target tissue.

22. The method of claim 21, further including, prior to the step of advancing the suction dome toward target tissue, the steps of:
longitudinally translating the suction dome in collapsed form through the suction channel of the instrument; and
deploying the suction dome.

23. The method of claim 21, further including the step of operably inserting a needle through the suction dome and permeable membrane to puncture, irrigate, and/or aspirate the target tissue.

24. The method of claim 23, further including the step of dissecting, or freeing, the target tissue from surrounding tissues while the target tissue remains in reliable contact with the suction dome.

25. The method of claim 21, further including the step of dissecting, or freeing, the target tissue from surrounding tissues while the target tissue remains in reliable contact with the suction dome.

26. The method of claim 25, further including:
collapsing the outer membrane of the suction dome around at least part of the target tissue, while maintaining reliable contact with the target tissue; and
withdrawing the suction dome and tissue through the instrument.

27. The method of claim 24, further including:
collapsing the outer membrane of the suction dome around at least part of the target tissue, while maintaining reliable contact with the target tissue; and
withdrawing the suction dome and tissue through the instrument.
